(19) United States
(12) Patent Application Publication
(10) Pub. No.: US 2016/0184049 A1
(43) Pub. Date: Jun. 30, 2016

(54) Non-Contact Sterilization Catheter

(71) Applicant: Vanguard AG, Berlin (DE)

(72) Inventor: Hagen Thielecke, Neuenhagen (DE)

(21) Appl. No.: 14/974,056

(22) Filed: Dec. 18, 2015

(30) Foreign Application Priority Data
Dec. 19, 2014 (DE) 102014119262.1

(51) Int. Cl. A61B 90/70

(52) U.S. Cl. CPC A61B 90/70

(57) ABSTRACT

Cleaning system for cleaning at least one region of a medical device that can be brought in contact with a human or animal body.

Means (14) for positioning the medical device in the cleaning system are present, such that the region that can be brought in contact with the human or animal body is positioned in a non-contact manner in the cleaning system.

The present invention relates to a cleaning system for cleaning at least one region of a medical device that can be brought into contact with a human or animal body.

EP 1 450 890 B1 discloses an apparatus for preparing balloon catheters, in particular cardiac catheters.

The apparatus for preparing balloon catheters comprises essentially an external ultrasonic pan. In addition, an internal holding pan is also provided and is arranged in the ultrasonic pan. In addition, a screen insert is arranged in the holding pan to hold medical devices, in particular balloon catheters.

A spray nozzle arrangement is provided inside the cover.

In addition, there are known medical devices consisting or a grip part or connecting part and a long flexible and tubular part. The medical device is inserted into a human or animal body for use.

One example of such a medical device is a cardiac catheter.

Standard cleaning and disinfection devices have so far been used for machine cleaning of long and/or tubular medical objects, such as catheters.

The medical devices are usually rolled up for cleaning and are introduced into a load carrier of the standard cleaning and disinfection device.

In addition, the medical device is designated as a catheter as an example but by no means exclusively.

When the catheter is inserted as described above into a standard cleaning and disinfection device, this leads to the following disadvantages.

First, a radius can be impressed into the catheter in rolling, so that the property of the medical device can be altered.

In addition, some regions of the catheter lie on the load carrier. It is also conceivable for the catheter to be covered at least in part by a spacer or some other holding device.

In the regions of the catheter lying on the load carrier or covered by holding devices, a spray jet with cleaning agent does not strike the surface of the catheter unimpeded.

One problem is that, the catheters lie on a carrier, which is why patient contact regions of the catheter are also shaded during the cleaning process, so they are not accessible to the cleaning process.

As a result, the covered regions of the catheter are cleaned poorly or not at all in comparison with the regions where the spray jet can strike the surface unhindered during the cleaning process.

This is especially critical when the covered regions of the catheter are regions, which are inserted into the patient in a sterile or almost sterile environment, namely the so-called patient contact regions.

The covered regions of the catheter must also be cleaned by a manual method.

In this context, the term “cleaning” is also to be understood to include disinfection and/or sterilization.

Therefore, an additional cleaning of a covered region of a catheter is an increased effort and expense. It is also very difficult to verify the efficacy of the additional cleaning.

The cleaning of catheters in standard cleaning and disinfection apparatuses often results in the catheter being rolled up, during which it may be bent.

The object of the present invention is therefore to provide a cleaning system for cleaning medical instruments having open lumens or no open lumens, in which no regions of the medical device (e.g., catheter) remain free, i.e., unwetted, by a cleaning agent during the cleaning process.

This object is achieved with a cleaning system, a cleaning method and use of the cleaning system according to the features of Claims 1, 17 and 20.

The load carrier serves to receive and position the medical instrument during cleaning. The load carrier is preferably arranged in a housing of the cleaning system, so that It is movable on guide rails.

The device for changing the alignment of its application region with respect to the cleaning agent is arranged so that it is movable by means of a guide mechanism of the load carrier.

The term “application” is understood to refer not only to application of medical instruments or wetting of medical instruments with a liquid, but instead it refers to any application of a fluid or other agent for cleaning, disinfection or sterilization. To change the alignment of the application region of the medical instrument with respect to the cleaning agent, the medical instrument, for example the catheter, is fastened to and hung on its gripping region and/or its connection region.

The patient contact region of the catheter is in this way bringing down freely into the cleaning pan.

It is also conceivable tor the guide mechanism to preferably be a hook, on which the catheter is hung with its grip by which the operator operates the catheter.

In contrast with the prior art, this achieves the result that the catheter, following the force of gravity, hangs down in an elongated orientation from the guide mechanism of the load carrier.

The guide mechanism is designed so that the patient connecting region of the catheter is only in loose contact with the guide mechanism.

In addition, it is conceivable for the position of the patient connecting region to be altered due to a change in the alignment of the application region by the guide mechanism during the cleaning process, preferably with respect to the spray jets.

In this way, a static shading of a region of the medical device, preferably the patient contact region, is prevented during the cleaning process.

Another advantage is that, because of the guide mechanism of the cleaning system according to the invention, non-contact or at least minimal-contact cleaning or disinfection and sterilization of the contact region of the catheter is/are possible.

The medical instrument, preferably the catheter, often has a continuous interior lumen and a corresponding exterior circumference. The cleaning system therefore preferably has flushing channels to also enable cleaning of these lumens and catheters having continuous open lumens.

The flushing channels of the load carrier are preferably connected to the lumen of the catheter by means of a coupling mechanism.

The flushing channel may be operated with a high water pressure or air pressure for cleaning purposes.

The lumens of the catheter are preferably connected with the help of tubes integrated into the load carrier and connected to the coupling mechanism between the load carrier and the cleaning system.

The lumen, i.e., the interior or the inside clearance of the medical device, preferably a catheter, which is hollow in particular, is open end continuous.

A plurality of individually controlled valves is preferably provided for supplying the cleaning agent. Flowmeters which determine the flow of cleaning agent through each coupling mechanism are preferably connected in series for each individual valve.

The flow of cleaning agent can be measured individually at all connections in a test operation. If the flow is not measured or if it differs greatly from the ideal value, the respective valve can be switched off.

All the connections for the defining agent flow are connected to a supply branch and to a drain branch.

The cleaning system is preferably installed in a wall frame.

Additional components of the system for cleaning medical equipment, such as pumps, valves, pipelines, etc., are arranged behind a control cabinet. The preceding list is given only as an example and is by no means intended to be exclusive.

Because of the arrangement behind the control cabinet, the aforementioned components are readily accessible for maintenance work in particular.

The cleaning system is mounted so that it can be moved and/or shifted and/or pivoted and/or rotated, preferably on a rail system.

A serving carrier that can be connected directly to the cleaning system is provided.

With the help of the serving carrier, the cleaning system can be completely separated from a wall rack and extracted from its resting position.

Access to the components of the system described above is further simplified in this way.

The serving carrier is designed to be portable and can be stored separately when not in use.

The cleaning agent may be a fluid and may be in liquid form. Purely as an example, which is by no means intended to be exclusive, media such as ozone, water, ultra sound, UV radiation, oxygen, chlorine, thermal energy or combinations thereof may be used as the cleaning agent.

It is also conceivable for the cleaning agent to be in the form of energy, e.g., ultrasound,

Purely as an example and by no means intended to be exclusive, “Neodisher Septoclean” may be used as the cleaning agent for both sterilisation and disinfection.

The term “cleaning” also includes the term “neutralization.” For example, the cleaning agent “Neodisher Z” may be used for neutralization.

The inside diameter of the medical device, preferably the lumens of the catheters, are connected to the load carrier with the help of tubes.

The lumen of the catheter is connected with the help of tubes integrated info the load carrier and with the help of the coupling mechanism between the load carrier and the system for cleaning the medical device. A dosing cabinet is also provided, in which four storage containers are preferably arranged. At least one storage container can be filled with the respective cleaning agent from a central supply.

Release of the cleaning agent and/or refilling of cleaning agent can be regulated by means of filling level sensors in the storage tank.

The amounts of cleaning agent to be dosed for a supply from the storage container into the mixing pan can be determined gravimetrically and/or preferably with the help of at least one dosing pump.

Next to the storage container, a connection is provided for taking a sample. With the help of the sample, cleaning agent can be taken from the mixing pan or a cleaning pan and the amount or concentration of the cleaning agent can be re-measured externally.

A mixing pan is provided, in which the cleaning agent for disinfection and/or sterilization is prepared for further use in the cleaning pan.

In the mixing pan, the cleaning agent, preferably water for cleaning, can be heated using a thermostat. No further thermostatic control of the water for cleaning in the cleaning pan is required.

Three large heating elements are preferably installed in the mixing pan, each being activatable separately, depending on the filling level of the cleaning agent, in particular the water for cleaning. A uniform temperature of the cleaning agent and/or of the water for cleaning can be achieved in this way.

The filling level of the mixing pan can be monitored with the help of filling level sensors and/or pressure sensors.

Additional cleaning agents and/or disinfectants can also be supplied via the mixing pan.

Dosing pumps are provided, which remove cleaning agent from the dosing cabinet and conduct it to the mixing pan through additional connections.

To prevent overheating, preferably of the water for cleaning and for uniform temperature distribution, the water for cleaning can be removed continuously from the mixing pan through a drain and supplied to the circulation through a water return.

The mixing pan and its inlet and outlet lines can be made fluid-tight with sealing elements.

The cleaning, i.e., preferably the disinfection and/or sterilization of the medical device take place in a cleaning pan, which is also known as a catheter pan.

The mixing pan and the cleaning pan can be manufactured from strips of sheet, metal, which are welded tightly to one another to form the respective pan.

Together with the load carrier, the cleaning pan forms a significant part of the system for cleaning the medical devices.

The cleaning can take place here, with or without an additional ultrasonic bath.

Ultrasonic sonotrodes are additionally arranged in the cleaning pan. The sonotrodes are arranged on at least one outside wall of the cleaning pan.

At least one heating mat is fastened on the side of the cleaning pan opposite the ultrasonic sonotrodes.

The heating mat regulates a continuously dropping water temperature in the cleaning pan. In addition to or as an alternative to the heating mat, at least one continuous heater is provided.

The at least one heating mat and the sonotrodes are preferably distributed over three height levels.

In this way, the plant, for cleaning the medical device can be operated separately in three different cleaning agent filling levels.

When using medical devices of shorter lengths and when using an ultrasonic bath, it is possible to save on a great deal of water for cleaning.

Filling level sensors, with the help of which the filling level of the water for cleaning in the cleaning bath can be determined, are provided in the cleaning bath.

The cleaning bath can thus be prevented from running empty. At the same time, damage to the at least one sonotrode during operation above the water level can be prevented.

Use of filling level sensors has also proven advantageous because the at least one heating mat must not be operated without an adequate filling level of water for cleaning.

In addition, it is conceivable to also measure the filling level of the water with the help of a pressure sensor in the drain of the cleaning pan.

The cleaning pan can be widened to prevent spray shadows caused by the arrangement of a protective chamber in the area of the coupling mechanism.

Spray jets and/or an ultrasonic bath can be used for external cleaning of the medical device.

During operation of the system for cleaning medical devices, not only are catheters preferably cleaned but also the load carrier itself as well as the entire interior space of the cleaning bath may be cleaned.

The spray nozzles used for this purpose fulfill the additional function of circulation and filtration of the water for dealing.

A filter corresponding to the filter used in cleaning the interior lumen is provided for this purpose.

The recycled cleaning agent or water for cleaning can be introduced into the mixing pan through a rotating spray nozzle. In this way, the interior of the mixing pan can be rinsed separately.

The spray nozzles are preferably subdivided into a catheter region and a coupling region, depending on the arrangement.

The cleaning pans or catheter pans are known to be designed to be comparatively small.

It is conceivable to use spray nozzles with a 90° spray jet to nevertheless achieve a large spray jet area in the region of the suspended catheters.

These spray nozzles are aligned along the direction of the wall of the cleaning pan.

In this way, the spray jets have a longer path until they strike the next surface. The impact surface is also increased accordingly.

A total of 24 spray nozzles, which can be operated at the same time or with a time offset, are preferably installed.

The spray nozzles are distributed over the total wall height of the cleaning bath.

The spray jets are arranged with different filling levels, some of them above the water level of the ultrasonic cleaning, so that they also reach the exposed surfaces of the inside surface of the cleaning pan.

To prevent a shaded area from the ultrasound, the spray jets are arranged only on one pan side of the plant for cleaning. Ultrasonic sonotrodes are also provided on the opposite side of the spray nozzles.

Because of the narrow wall spacing inside the cleaning pan, the spray jets may be reflected strongly on the respective wail surfaces and thus arrive at each point along the inside contour of the cleaning pan.

In the region of the coupling mechanism, it is provided that, in addition to reaching the load carrier, the spray jets also reach the regions above the load carrier and the coupling mechanism.

It is conceivable for the cleaning pan to be filled manually to approximately ⅕ of the pan height, to thereby subject the spray nozzles to a test.

When using a pump and the respective valves, the circulation of the cleaning agent causes the spray jets to reach each region Inside the cleaning pan.

Use of a high spray jet pressure and a small distance between the surfaces of the inside contour of the cleaning pan results in reflection of the jets emitted by the spray nozzles.

In this way, the application density of the cleaning agent, in particular the spray mist density, can be increased further.

In a test run, the cleaning pan can be filled up to the maximum height to start operation of the ultrasonic sonotrodes.

At full power, operation of the ultrasonic sonotrodes leads to formation of bubbles in the interior of the cleaning pan.

It can be seen from this that the applied ultrasound can be transferred through the cleaning pan into the water for cleaning.

To create a higher cleaning pressure, thorough flushing of the interior lumen of the catheter should preferably take place. After this, the exterior cleaning of the catheter is performed with the help of the spray nozzles.

A protective chamber, in which catheter grips and coupling devices of a catheter, preferably arranged vertically in the cleaning system, are held, is also provided. In particular, the catheter grips with the electronic controllers of the catheter arranged therein are arranged in the protective chamber and held there, so that they are protected from water for cleaning and the cleaning chemicals.

The at least one protective chamber is arranged in the upper region of the load carrier of the cleaning system.

The arrangement of a protective chamber has proven to be extremely advantageous because the catheter grips are protected from the cleaning chemicals of the cleaning agents.

With the help of a transfer station, the load carrier can be removed from the system for cleaning medical devices and may also be reused.

The loading of the load carrier, preferably with catheters, can in this way take place outside of and independently of the system for cleaning the medical devices.

The arrangement of a protective chamber has also proven to be highly advantageous because in this way preferably the electronics of the medical device, which are arranged in the grip region of the device in particular, can be protected from cleaning agent during the cleaning.

It is also conceivable for the protective chamber to be used instead of the coupling mechanism.

The assembly of the protective chamber with grips for the catheter in particular takes place by inserting the grip of the respective catheter into the protective chamber. The catheter shaft connected to the catheter grip with its patient contact region preferably protrudes into the cleaning pan in an elongated and essentially vertical fashion.

It is conceivable for the shaft of the catheter to hang down into the cleaning pan, preferably through a slot the bottom of the protection chamber, because of the force of gravity. The chamber bottom then protects the protective chamber from penetration of water for cleaning and/or cleaning agent. The grip of the catheter in particular is subjected to a separate cleaning and/or disinfection before being inserted into the protective chamber.

Spray shadows that may be formed due to the use of a protective chamber can be compensated and/or minimized by an enlarged spray angle of the spray nozzles. It is conceivable to provide preferably 12 spray nozzles with a total spray angle of 120° to minimize the occurrence of spray shadows.

A housing to hold the load carrier comprises a frame carrier, which is a main structure of the load carrier.

The coupling mechanism and the at least one guide device are mounted on the frame carrier.

The housing with its frame carrier comprises at least two swivel doors hung on the frame carrier.

The doors of the housing may be opened for assembly of the load carrier with catheters and/or for removal thereof from the load carrier. The at least one door of the housing can preferably be closed after the end of the assembly of the load carrier.

After assembly of the catheter on the respective extension pipe of the load carrier, it is provided that the shaft of the catheter is threaded through a hook on the carrier frame. Threading the catheter through the hook reinforces the vertical alignment of the catheter in particular.

This has proven to be extremely advantageous because in this way the shaft of the catheter is limited in its mobility. The catheter cannot fly around in an uncontrolled manner while the inner lumen is being cleaned with high water pressure or air pressure.

The at least one door is provided with a door gasket, which preferably prevents water for cleaning and/or cleaning agent from escaping during the cleaning.

It is conceivable to use inflatable profile gaskets, which can be welded over a miter cut.

Additional sealing elements may be arranged in the coupling region between the cleaning system and the load carrier as well as in the region of the mixing pan and/or the cleaning pan.

A coupling mechanism, in which the coupling for the cleaning agent and/or the water for cleaning is/are preferably provided between the cleaning system and the load carrier, is preferably provided at the upper end of the load carrier.

The connection for the catheter can preferably be extended downward by means of at least one extension pipe.

This achieves the result that the continuous inside volume of the catheter can preferably be flushed with water for cleaning and/or cleaning agent.

It is conceivable for the extension pipe to be arranged in two rows of 15 connections each.

In addition, it is conceivable for the coupling mechanism to have a modular design and to be completely removable and interchangeable with a protective chamber in this way.

In addition, guide rollers are provided, facilitating the loading and unloading of the load carrier from the cleaning system onto and from a shipping car. Notches may be provided so that the load carrier engages in the intended position.

It is conceivable for the coupling mechanism to preferably include 30 compressed air cylinders with a plunger mounted on each.

The plungers are moved down to the load carrier by cylinders and can be coupled to the load carrier. Tubes which can be charged with water for cleaning are provided on the side of the plungers.

All the plungers are operated at the same time.

A coupling in the region of the load carrier has two other cylinders which can be controlled separately.

Two other cylinders preferably serve to center and/or position the load carrier in the cleaning system prior to the mechanical coupling for the supply of water for cleaning.

The catheters can be flushed with water for cleaning in this way.

The water for cleaning and/or cleaning agent that flows through the catheter is received in the pan bottom of the cleaning pan and can be sucked out of the cleaning pan.

It has proven advantageous that the used water for cleaning can be reused repeatedly for cleaning and/or disinfecting.

Schematic Diagram of the system

The plant layout of the cleaning system is summarized and simplified according to the diagram shown in the drawing.

The arrows show which modules are associated with one another and in which direction the cleaning agents and/or the water for cleaning are replaced.

The water for cleaning 1 is added directly to the mixing pan 2 and cleaning agent 3 also is added.

On reaching the desired temperature and the intended filling of the mixing pan 2, the cleaning agent 3 is transferred from the mixing pan 2 into the cleaning pan 4.

WFI water (osmosis water) 5 is used for the final rinse 6 and then is not introduced into the cleaning pan 4 through the mixing pan 2 but instead is introduced directly.

The WFI water 5 is circulated repeatedly for rinsing the outer and inner lumen.

In the case in which the required conductivity of the WFI water 5 is not reached, the final rinsing 6 with WFI water 5 must be performed again.

Sterile air 7 is used once for the mechanical control 8. For this purpose, the region of the coupling mechanism 9 between the cleaning system and the load carrier 14 is used for media connections, the gasket on the door and pneumatic valves in particular.

Preferably clean air 7 is blown Into the two pans 2; 4 in the emptying 11 of the WFI water 5 into the two pans 2; 4. In this way, the ambient air cannot flow into the respective pan 2; 4 during the emptying 11.

During operation of the cleaning system, WTI water samples 12 can be taken from the respective pans 2; 4 in each operation, as soon as this is allowed by the control technology.

A conductivity measurement 13, which is provided here, can be verified with an external measurement device.

Disposal: Water can be sent directly to the drain 11 from the cleaning pan 4 and the mixing pan 2.

A pump 10 is provided for this purpose. It is also conceivable to ensure the outflow through a free drain.

Mixing pan: The mixing pan 2 serves to prepare the WFI water 5 with or without cleaning agent 3 for the cleaning pan 4.

The cleaning and/or disinfection and/or sterilization of the medical devices 13 take(s) place in the load carrier 14 and in the cleaning pan 4.

Test of Cleaning of Egg Yolk Soiling

Soiling in the form of dried egg yolk was introduced into the cleaning system.

This test cleaning was used to investigate the results achieved by spray nozzle cleaning and ultrasonic cleaning.

Egg yolk was introduced as the soiling into the cleaning system and dried there.

For visual enhancement, the water-soluble sodium salt of fluorescein uranium was added to the egg yolk.

The entire load carrier and the internal contour of the cleaning pan were soiled in all the spray shadow regions in this test.

The drying time was approximately 3 days.

After adequate and extensive drying of the egg yolk for several days, operation of the cleaning system was started with the load carrier.

The cleaning was first performed with spray jets.

In doing so, the egg yolk was released very rapidly by the impinging spray mist.

Egg yolk residues remained only in the spray shadow regions and in gaps where there was heavy soiling with egg yolk.

The ultrasonic hath was used next.

In doing so, the cleaning pan was filled with water and additional egg yolk residues were dissolved in the water.

An intense discoloration of the water occurred when the ultrasound was turned on.

The residues dissolved out of the shadow region in the pan and in the gaps.

Following that, the water was pumped out and the pan was rinsed with clean water.

At the height of the coupling mechanism, at least one spray nozzle was replaced by a ball joint spray nozzle. The ball joint spray nozzle could be directed at the at least one door.

It is also conceivable to prevent additional residues, primarily on the load carrier, by beveling the edges.

Result After the Test Cleaning

The cleaning pan and the mixing pan were cleaned to great satisfaction by the test cleaning for the visual impression.

Dirt particles were completely removed with a sufficient supply of water for cleaning. The dirty water was able to how out due to gravity.

Visualisation of the System

It is provided that a display screen is to be set up on the control cabinet. All the activated components in the cleaning system are identified in color during use.

In addition, the values such as pressure and temperature measured by the sensors are displayed simultaneously.

The current measured values and the activated components can be displayed during the entire cleaning process.

A program sequence according schedule is displayed.

In another embodiment, visual buttons can be placed on a display screen, so that functions such as an emergency OFF switch and door opening can be triggered.

In addition, it is also possible to switch between different displays, such as the temperature profile and the pressure profile, during the cleaning process.

Visualization of the Flow Test

On activation of the cleaning system, a test is performed to ascertain whether the flow of cleaning agent and/or the flow of water for cleaning is/are measured.

If the flow is determined satisfactorily, the connections for the cleaning agent and/or the water for cleaning are released and labeled in color.

If there is no determination of flow because of defective valves or blockage of a catheter, the supply connections can be blocked and can also be displayed in color.

Individual valves can be deactivated manually.

Visualization of the Measured Values

The values such as pressure and temperature measured by the sensors can be displayed graphically in real time on the display screen.

The behavior of the cleaning system can be investigated over the entire batch on the basis of a curve for the data thereby determined.

Reference Numerals

1 water for cleaning

2 mixing pan

3 cleaning agent

4 cleaning pan

5 WFI water (osmosis water) 5

6 final rinse

7 sterile air

8 mechanical control

9 coupling mechanism

10 pump

11 device for emptying the pan

12 water sample

13 conductivity measurement

14 load carrier

1. A cleaning system for cleaning a least one region of a medical device that can be brought into contact with a human or animal body, characterized in that means (14) are present in the cleaning system for positioning the medical device so that the region that can be brought into contact with the human or animal body is positioned in the cleaning system in a non-contact manner.

2. The cleaning system according to claim 1, characterized in that the device for positioning in the cleaning system is held in a region that cannot be brought into contact with the human or animal body.

3. The cleaning system according to claim 2, characterized in that the device for positioning in the cleaning system is secured on a grip or on a hook.

4. The cleaning system according to claim 1, characterized in that the device is arranged in loose contact with the positioning means (14) in the cleaning system.

5. The cleaning system according to claim 1, characterized in that the position of the region that can be brought in contact with the human or animal body can be altered in comparison with a device for applying cleaning agent.

6. The cleaning system according to claim 1, characterized in that the means for positioning the device is a load carrier (14), which has at least one coupling mechanism (9) that supplies the cleaning agent (3).

7. The cleaning system according to claim 6, characterized in that the coupling mechanism (9) is arranged on the end of the medical device facing the load carrier (14).

8. The cleaning system according to claim 6, characterized in that the coupling mechanism (9) is arranged on the end of the medical device facing away from the load carrier (14).

9. The cleaning system according to claim 1, characterized in that the means (14) for positioning the device has at least one extension pipe for connecting the device. cm 10. The cleaning system according to claim 1, characterized by a housing in which the means (14) for positioning the medical device is arranged so that it can be displaced.

11. The cleaning system according to claim 10, characterized in that the housing can be closed by at least one door.

12. The cleaning system according to claim 1, characterized in that the cleaning system has at least one rinsing channel and/or at least one spray nozzle.

13. The cleaning system according to claim 1, characterized in that a dosing cabinet is arranged for supplying the cleaning system with cleaning agent (3).

14. The cleaning system according to claim 1, characterized in that cleaning pan (4) that holds the cleaning agent (3) is provided.

15. The cleaning system according to claim 14, characterized in that at least one sonotrode and/or at least one heating mat is/are arranged in the cleaning pan (4).

16. The cleaning system according to claim 1, characterized in that a mixing pan (2) is provided for preparing the cleaning agent (3) for application.

17. The cleaning system according to claim 1, characterized in that at least one filling level sensor is provided for controlling the filling level of cleaning agent in the cleaning bath (4).

18. The cleaning system according to claim 1, characterized in that a protective chamber is provided for protecting a region of the medical device that cannot be brought in contact with a human or animal body.

19. A method for cleaning medical devices with the cleaning system according to claim 1, characterized in that the region of the medical device that can be brought in contact with the human body is positioned in the cleaning system in a non-contact manner with the help of at least one positioning means (14).

20. The method for cleaning medical devices with the cleaning system according to claim 19, characterized in that, prior to being introduced into the cleaning pan (4), the cleaning agent (3) is prepared in a mixing pan (2) for the cleaning process, and sterile air (7) is used to control (8) the process.

21. The method according to claim 19, characterized in that at least one cleaning agent sample (12) is taken from the cleaning pan (4) and/or the mixing pan (2).

22. A use of a cleaning system according to claim 1 for cleaning surgical medical devices in gynecology or obstetrics or otolaryngology or ophthalmology or dentistry or urology or trauma surgery.
